(19) United States
(12) Patent Application Publication
(10) Pub. No.: US 2007/0009450 A1
(43) Pub. Date: Jan. 11, 2007

(54) Preparation, in particular cosmetic preparation, process for the...

(76) Inventor: Susanne Emig, Nuernberg (DE)

Correspondence Address: BACHMAN & LAPOINTE, P.C., 900 CHAPEL STREET, SUITE 1201, NEW HAVEN, CT, 06510, US

(21) Appl. No.: 10/555,040

(22) PCT Filed: Aug. 31, 2005
(86) PCT No.: PCT/EP05/09388

§ 371 Date: Nov. 21, 2005

(30) Foreign Application Priority Data
Sep. 3, 2004 (DE) 10 2004 042 802.6

(51) Int. Cl. A61K 8/19; A61K 8/35; A61K 8/37

(52) U.S. Cl. 424/59; 424/63; 424/64; 977/926

(57) ABSTRACT

A preparation, in particular a cosmetic preparation, in the form of a workable paste which is present in the form of an emulsion and which is suitable for cosmetic uses, in particular in the area of decorative cosmetics for colouring and improving the skin, the lips and the eyelids. The preparation is also suitable as a fixing for lipstick, as lip care, as a skin care foundation or as a sun protection agent. The preparation contains an ester component, a suitable emulsifier, a volatile silicone oil, optionally a moistening agent, a solids phase and water. addition the preparation may also contain the additives and adjuvant substances which are approved and usual in cosmetics. Processes for the production of the preparation are described.

### CROSS REFERENCE TO RELATED APPLICATION(S)

This application is a continuation-in-part application of U.S. patent application Ser. No. 11/189,255, filed Jul. 26, 2005, entitled PREPARATION, IN PARTICULAR COSMETIC PREPARATION, AND THE PRODUCTION AND USE THEREOF, By Susanne Emig et al.

### BACKGROUND OF THE INVENTION

The invention concerns a preparation in the form of an emulsion comprising a lipophilic outer phase and a hydrophilic inner phase, in particular a cosmetic preparation, as well as the production and use thereof.

Emulsion-like preparations are used in many areas, in particular in the field of cosmetics and foodstuff chemistry. Emulsions are heterogeneous systems comprising two phases which are not miscible with each other or not unlimitedly miscible with each other, an inner phase being distributed in finely divided form in the other outer phase. The outer phase which contains the inner phase in the form of fine to very fine droplets is referred to as the continuous phase and determines the basic character of the emulsion. Emulsions in which water forms the inner phase have advantageous properties. Inter alia microbiological problems are less likely to occur with that kind of emulsion.

The outer phase used for such emulsions is for example lipids such as oils or fats, or silicones. The silicone can be volatile or non-volatile and it can be silicone oil, silicone resin, silicone gel or a silicone copolymer.

In order to keep the emulsion system stable over a prolonged period of time and to prevent the droplets of the inner phase from flowing together and settling as a phase, it is necessary to add emulsifiers which have a hydrophilic and a hydrophobic component and which are deposited around the droplets.

Emulsions which are frequently used in the cosmetics field, with an outer oil phase, so-called W/O emulsions, often suffer from the disadvantage that the outer lipophilic phase leaves behind after application on the skin a greasy or sticky feel which is perceived as being unpleasant. That unpleasant effect can also occur when using silicones as the outer phase. When using such emulsions in the region of the eyes, there is also the disadvantage that the oil or silicone phase migrates into the eye or the conjunctival sac and results in an oily veil on the lens and unpleasant irritation effects.

A disadvantage with the known preparations is that they can easily transfer from the skin or also the lips to which they have been properly applied onto other surfaces, for example onto cups, glasses, textiles or also other areas of the skin. That can leave behind traces in the form of a coloured impression or a film of fat. Such products therefore have inadequate adhesion to the surface to which they are applied, with the result that lip rouge and blusher, makeup, eyeshadow and also sun protection agents have to be regularly re-applied. As oily constituents generally spread very well on the skin and the lips, the pigments migrate from the original place at which they were applied together with small amounts from the oil phase into the fine wrinkles of the skin in the immediate surroundings, which often has a very detrimental and adverse influence on the overall visual impression.

In the past, the attempt was made to combat that in regard to lipsticks and lip rouges by the use of so-called “bromo acids”, dyes which are substantively absorbed onto the skin. As however different pH-values of the skin meant that those dyes gave individually different and unpredictable shading effects and in addition the colouring often persisted for days, that route was quickly abandoned again because such products met with only inadequate acceptance on the part of lady consumers.

After silicone oils and silicone resins had found their way into cosmetics, the attempt was made to improve the adhesion to the skin and thus the durability of decorative preparations in that way. Thus, eyeshadow pencils and lipsticks have been known from about 1977, in the form of leads which are cast into sharpenable casings which included in the lipid phase inter alia a mixture of phenyltrimethicone (a non-volatile silicone oil) and cyclomethicone (a volatile silicone oil). They were then followed by similar preparations which contained cyclomethicone as the sole silicone component. Although they had an apparently firm structure, those pencil materials could be applied to the skin in a soft smooth and workable fashion, similarly to a pasty material. Materials of that kind are described in numerous applications, for example in DE 27 18 957, DE 27 59 610, DE 27 59 856 and DE 30 28 231 and can be produced with the processes described therein. In addition DE 40 05 894 describes a method of filling pencil materials of that kind.

After evaporation of the volatile silicone what remained was a soft elastic film on the skin, which had very good adhesion thereto and which involved only minimal migration into the area around the original place of application. The principle of the combination of two silicone oils, transferred to pasty preparations, is also to be found again for example in EP 0 756 864.

Although silicone oils in those preparations of the previously known kind were able to improve adhesion and durability, the silicone oils, in relation to sensitive users, gave rise to detrimental effects if they were used in the immediate proximity of the eye. More specifically, if even minimal amounts of silicone oils, in particular non-volatile silicone oils or other silicone polymers, from the products in stick form known from the state of the art, get into the eye or the conjunctival sac, they can result in an oily veil on the lens and unpleasant irritation effects, referred to as the “wind burn effect”.

A further problem arises in regard to emulsions which contain particulate or finely divided ingredients such as pigments or effect substances. The particles can destabilise the emulsion and settle so that the result is an aesthetically unsatisfactory two-phase composition.

Therefore the object of the invention was to provide a preparation, in particular a cosmetic preparation, which does not suffer from the disadvantages of the products of the state of the art, and which is in the form of a workable paste. A further object was to provide a preparation which remains stable over a prolonged period of time and in which the phases do not separate, even if it contains particulate ingredients.

A further object of the invention was to provide a storage-stable composition which does not separate into phases even upon prolonged storage at different temperatures in different climatic zones. The invention also aims to provide that the preparation can be well applied and adheres to the place of application and after drying neither migrates into the surrounding area nor transfers onto other surfaces.

Another object of the invention was to provide a preparation which can be produced free from substances derived from animals, that is to say which contains exclusively ingredients from plants and/or mineral and/or synthetic materials.

Still a further object of the invention was to provide a preparation which shows off to good advantage effect materials and causes them to gleam, without producing a disturbing “fat shine”.

### SUMMARY OF THE INVENTION

Those objects are attained with a preparation according to the invention in the form of an emulsion with a lipophilic outer phase and a hydrophilic inner phase, wherein the outer phase contains an at least divalent ester component, a volatile silicone, an emulsifier, a particulate phase and optionally a moistening agent and further ingredients which are usual in cosmetics, and the inner phase has an aqueous medium and optionally hydrophilic additives which are usual in cosmetics, wherein the ester component has a melting point in the region of 40 to 200° C.

### DETAILED DESCRIPTION

It has been surprisingly found that the preparation of the present invention does not suffer from the disadvantages of the state of the art. In particular it remains stable over a prolonged period of time, enjoys excellent adhesion to the place where it is applied, it is wipe-resistant and involves practically no transfer onto other surfaces such as cups, glasses, textiles or also other areas of the skin. It is also does not leave behind any traces in the form of a coloured impression or a film of fat. As it does not contain any oily constituents which spread on the skin and the lips, the pigments does not migrate from the place of application into the fine wrinkles of the skin. The preparation according to the invention therefore affords excellent visual results. The preparation has a pronounced structural viscosity and can therefore be applied easily and malleably and in different layer thicknesses so that after application it is possible to form soft and gentle transitions to the untreated skin. In contrast to known silicone-bearing compositions, it does not cause the skin to dry out and it also does not produce any tautening feeling. The preparation according to the invention, after drying, neither migrates on the skin nor into the wrinkles of the lips. In spite of the content of silicones it does not tauten and dry out the skin. The storage stability at the different temperatures of the different climatic zones is excellent and thus satisfies statutory requirements. Even after a prolonged storage time it does not exhibit any syneresis effects.

The preparation according to the invention is present as an emulsion in the form of a workable paste and is suitable for cosmetic uses, in particular in the field of decorative cosmetics, for colouring and improving the appearance of the skin, lips and eyelids. Mention may be made here by way of example of lip rouge and blusher, makeup, concealer, eyeshadow, agents for fixing lipstick or lip rouge, foundation care for care of the skin or sun protection agents.

The advantageous properties are achieved by a combination of selected ingredients. The ingredients are described in greater detail hereinafter and contribute the good properties. By using those ingredients, it is possible to dispense with the use of fatty or oily constituents, in particular those which are derived from animals. In addition the proportion of non-volatile silicones is reduced so greatly that the disadvantageous properties thereof do not cause problems.

It was found that, in the case of a preparation, in particular a cosmetic preparation in the form of an emulsion, the combination of volatile silicone oil, ester component, particle phase and water does not make any further oil components necessary, in order to obtain a stable product which enjoys good adhesion. It is essentially possible to dispense with low-viscosity, non-volatile silicone oils; they are used, if at all, only in harmless, quite small amounts below 5 percent by weight, for example in order to delay the preparation drying time and at the same time as an anti-foam agent and emulsion aid.

Dispensing with fatty and oily components and disadvantageous silicone compounds is made possible in that, in accordance with the invention, a combination of volatile silicones with special esters is used. That combination provides that the viscosity changes only immaterially even during a prolonged storage time.

The preparation according to the invention in the form of an emulsion has an inner and an outer phase. The inner or disperse phase is aqueous and comprises an aqueous medium in which the hydrophilic constituents are dispersed and/or possibly dissolved. The outer phase contains at least one polyvalent ester component, at least one volatile silicone and at least one particulate phase. In addition the preparation also contains at least one emulsifier and can additionally possibly also contain further additives which are usual in cosmetics.

The essential constituents of the preparation according to the invention provide that the result is a stable emulsion. For that purpose the preparation according to the invention contains a wax-like ester component in combination with a volatile silicone which provide for the structure, at least one emulsifier which stabilises the emulsion, and an aqueous medium as a dispersed phase. In addition the preparation advantageously contains at least one moistening agent for keeping it moist and at least one finely divided solids phase.

The aqueous medium comprises water and/or aqueous or water-miscible solvents. The aqueous medium can be for example a water-alcohol mixture, in which case the alcohol is preferably ethanol or isopropanol. The aqueous medium is preferably water.

As an essential constituent, the outer phase contains a polyvalent ester component which contributes to structure formation and which is used as a wax-like constituent. In accordance with the present invention polyvalent is used to denote at least divalent esters. In that respect, those compounds are defined as polyvalent ester components, which are made up of polyvalent (at least divalent) alcohols with at least two respective acid residues, polyvalent (at least divalent) acids and at least two respective alcohol residues or polyvalent alcohols and polyvalent acids, wherein the chain length of the residues originating from the alcohol and the residues originating from the acid is C₂ to C₆₀, preferably C₄ to C₆₀, particularly preferably C₂₀ to C₆₀.

The ester component is intended inter alia to perform the function of a wax in the preparation and therefore preferably is of a melting point in the range of between 40 and 200° C., preferably from 55 to 125° C.

In order to obtain an ester component with the specified melting point, the sum of the carbon atoms of alcohol and carboxylic residues should be in a range of 35 to 150, preferably 40 to 100. Both alcohol residue and also carboxylic acid residue can have a saturated or singly or multiply unsaturated, straight-chain or branched hydrocarbon component and may possibly also have still further substituents in the form of functional groups such as hydroxyl, carboxyl, amino, acid amide and ester groups, and the like.

Preferably the polyvalent ester component contains at least one compound of formula I: 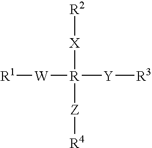
wherein R is a linear, branched or cyclic hydrocarbon residue with 1 to 8 carbon atoms, W, X, Y, Z are each independently of each other —C(O)O—,
—OC(O)—, —O—, —NR⁵₂ or —NC(O)— and each of the residues R¹, R², R³, R⁴ and R⁵ respectively independently denotes a linear or branched, long-chain hydrocarbon, preferably with 8 to 36 and particularly preferably 12 to 24 carbon atoms.

Particularly good properties are achieved if esters, in particular fatty acid esters of pentaerythritol, such as for example pentaerythrityl tetramyristate, tristearate, tetrastearate, triisostearate, tetraisostearate, tri-(12-hydroxy)-stearate, tetra-(12-hydroxy)-stearate, tribehenate, tetrabehenate, tetra-(ethylhexyl-dodecanoate), trierucate, tetraerucate, tetramelissinate and the like are used. It is also possible to use behenyl behenate, behenyl melissinate or isostearyl ethylhexyl dodecanoate alone or in mixture with pentaerythritol ester.

In addition to the ester component, it is also possible to use at least one vegetable wax, preferably a mixture of vegetable waxes, in particular a mixture of candellila wax and carnauba wax. Mixtures of the ester component according to the invention and waxes are possible, on the assumption that unwanted interactions between the individual components are to be minimised.

The ester components are used in an amount which is required to achieve the desired “zero viscosity”, corresponding to the desired purpose of use. Zero viscosity and complex viscosity can be determined with a rheometer of type MCR-301 from Anton-Paar (measuring system plate/plate, plates both sand-blasted, plate diameter 25 mm, gap width 1,000 μm; shearing rate at zero viscosity 0.00005 s⁻¹; temperature 298.15 K; software Rheoplus/32 V6.23).

In that respect the amount used is in a range of between 0.5 and 20 percent by weight, preferably in a quantitative range of between 2 and 12 percent by weight. It was surprisingly found that the ester components used in accordance with the invention, in particular the esters derived from pentaerythritol—possibly because of the three-dimensional spatial structure deriving from the tetrahedron model—are very well suited to gelling volatile silicone oils such as for example cyclomethicone or short-chain dimethyl polysiloxanes and in that way producing structures which facilitate application of the preparation according to the invention.

If in addition a wax is added to the preparation according to the invention, a preferred wax is one having a dropping point of between 50 and 200° C., in particular between 60 and 150° C., quite particularly preferably between 75 and 120° C. That additional wax component contains at least one alcohol residue of a chain length of between C₂ and C₆₀ and a carboxylic acid residue of a chain length of between C₄ and C₆₀. Both the alcohol residue and also the carboxylic acid residue can have a saturated or singly or multiply unsaturated, straight-chain or branched hydrocarbon component and optionally also still further substituents in the form of functional groups such as hydroxyl, carboxyl, amino, acid amide and ester groups, and the like. If used, the wax is employed in such an amount that the desired zero viscosity is achieve or maintained.

The second essential constituent of the preparation according to the invention is a volatile silicone oil. The term volatile silicone oil is used here to denote a silicone compound which is liquid at ambient temperature and which, when applied to the skin, that is to say at a temperature of between 30 and 38° C., evaporates slowly. The volatile silicone desirably is of a vapour pressure in the range of 0.1 to 20 kPa at 25° C., preferably 0.2 to 10 kPa and particularly preferably 0.5 to 5 kPa.

In accordance with the invention a preparation, in particular a cosmetic preparation, can be provided in the form of an emulsion which contains volatile silicone oils as the single liquid lipid component. Short-chain and cyclic dimethyl siloxanes are suitable. Examples in that respect are cyclomethicone such as hexamethyl cyclotrisiloxane, octamethyl cyclotetrasiloxane, decamethyl cyclopentasiloxane, dodecamethyl cyclohexasiloxane or short-chain dimethicones such as hexamethyl disiloxane, octamethyl trisiloxane, decamethyl tetrasiloxane, dodecamethyl pentasiloxane or mixtures thereof, wherein the amount thereof used is between 5 and 65 percent by weight with respect to the total weight of the preparation, preferably between 15 and 45 percent by weight.

The preparation may optionally contain non-volatile silicone oils in a small amount, for example as an anti-foam agent and emulsification aid. The amount of the non-volatile silicone oils is less than 5 percent by weight, preferably less than 1.5 percent by weight.

The disadvantages of preparations known from the state of the art, which contain oils and fats, can be overcome in accordance with the invention insofar as the volatile hydrophobic components used are only volatile silicone oil and optionally a small amount of non-volatile silicone oils which are used as anti-foam agents and emulsification aids. It was found that the combination according to the invention of ester component and volatile silicone oil achieves a very good level of consistency without the disadvantages entailed with lipid components.

The combination according to the invention makes it possible for a solid phase to be integrated stably in the preparation, which phase can comprise for example finely divided fillers, colouring agents and/or effect substances.

The above-mentioned solid or particulate phase comprises ingredients in particle form which do not dissolve either in the hydrophobic or in the hydrophilic phase. The particulate phase can comprise for example fillers such as for example talcum, kaolin, starch and modified starch, polytetrafluoroethylene powder (Teflon), nylon powder, boronitride, insoluble metal soaps such as Mg stearate, Ca stearate, Sr stearate, Zn stearate, effect materials such as sequins or spangles, glitter, fluorescent and phosphorescent particles and in particular inorganic or organic pigments or mixtures of the specified substances. The following may be mentioned as pigments by way of example: titanium dioxide, zinc oxide, iron oxides, chrome oxide, hydrated chrome oxide, ultramarine, Berlin Blue (Ferric Blue), micas, pearlescent agents such as for example micas coated with titanium dioxide, coloured micas coated with titanium dioxide and metal oxides, bismuth oxide chloride, coated bismuth oxide chloride, flake-form metal powder of aluminium, brass, bronze, copper, silver, gold and lakes of organic colouring agents with aluminium, barium, zirconium, calcium or strontium. That list is only by way of example and is not definitive.

In the case of sun protection agents, it is possible to use particularly finely divided particles, so-called nanopigments, of an average particle size of between 5 and 50 nm, which act transparently on the skin and no longer colour it. Mention may be made here by way of example of silicon dioxide, titanium dioxide, cerium oxide, aluminium oxide, zirconium oxide and zinc oxide.

In a preferred embodiment lakes of organic dyes are used as the colouring agents. It was found that the combination of lakes of organic dyes with the emulsion according to the invention provides that the dyes do not bleed out but remain in the structure produced.

Those additions are effected with the proviso that they are also allowed by the respective national or regional cosmetic legislation. The amounts used are preferably also within the limits of the maximum amounts allowed by the respective cosmetic legislation. The quantitative proportions of solids, in particular pigments, insofar as there are no statutory regulations preventing this, are in that case in a range of 0 to 40 percent by weight, preferably in a range of 5 to 30 percent by weight and quite particularly preferably in a range of 8 to 20 percent by weight.

In a further preferred embodiment, there is provided a preparation which contains pearlescent pigments and flake-form metal powders. It was surprisingly found that, in preparations according to the invention, if they contain colouring agents based on pearlescent pigments and flake-form metal powders, an unusually intensive sheen is produced which, without being bound down to a theory, is attributed to the fact that the pigment particles are oriented in the emulsion according to the invention, perhaps caused by the controlled evaporation of the volatile constituents silicone oil and water, and thus it is possible for them to be oriented to active charge centres of the skin.

At least one emulsifier is used to form the emulsions according to the invention. It is also possible to use a mixture of emulsifiers. Preferably an emulsifier which is suitable for W/S emulsions or a combination of a W/S and a W/O emulsifier is used. It is possible to add the emulsifiers which are known to the man skilled in the art. Preferably a non-ionogenic emulsifier is used. Preferred emulsifiers are those which are selected for example from sorbitan sesquioleate, sorbitan laurate, polyglyceryl-4 isostearate, PEG-5 soya sterol, soya sterol, polyglyceryl-2-PEG-4 isostearate, polyglyceryl-2 sesquiisostearate or cetyl-PEG/PPG dimethicone, such as for example cetyl-PEG/PPG-10/1 dimethicone. Experience has shown in that respect vegetable sterols such as soya sterols are preferably suitable as auxiliary emulsifiers. It will be appreciated that in principle it is also possible to use mixtures of the above-indicated emulsifiers—on the premise that unwanted interactions between the individual components are to be minimised however preferably only one of the above-indicated emulsifiers is used for producing the emulsion according to the invention. In principle however phosphate esters are also suitable as emulsifiers for production of the preparation according to the invention such as for example trioleyl phosphate, trioleth-8-phosphate or trilaureth-4-phosphate.

The required amounts of emulsifiers used are in the range of between 0.5 and 10 percent by weight, preferably between 1.5 and 6 percent by weight. For stabilisation of the emulsions according to the invention, it is optionally possible to add to the water phase inorganic salts which are easily soluble in water or salts (almost insoluble in water) of fatty acids, such as for example magnesium sulphate, sodium sulphate, sodium chloride, potassium chloride, magnesium stearate or magnesium myristate. The two last-mentioned components are to be carefully dispersed in the water phase prior to emulsion formation—they can however preferably also be mixed with the solids phase. The above-mentioned salts are used in amounts of from 0.05 to 3 percent by weight, but preferably from 0.3 to 2 percent by weight in the water phase.

In addition it is also possible to add to the preparation according to the invention, preferably in the aqueous phase, at least one moistening agent such as for example propylene glycol, dipropylene glycol, tripropylene glycol, butylene glycol, glycerine, diglycerine, triglycerine, sorbitol, mannitol, xylitol, glucose, fructose, sucrose, carbamide (urea), lactic acid, citric acid, pyrrolidone carboxylic acid (PCA) or the salts of the specified acids or mixtures of the specified substances. Preferably in that respect the sodium salts which are easily soluble in water are used. The amounts used are from 0.1 to 5 percent by weight, preferably from 1 to 3 percent by weight.

For stabilisation over a relatively wide temperature range, it is possible to use silica (amorphous silicic acid), bentonite, hectorite, montmorillonite or the like.

In addition, the preparation according to the invention may contain additives which usual in cosmetics such as preserving agents, antioxidants, fragrances, vitamins, sun protection filters and the like in the amounts usual for such substances.

The preparation according to the invention is distinguished in that the viscosity changes only immaterially even during a prolonged storage time. A particular advantage of the preparation according to the invention is that even ingredients of greater specific weight, for example particulate materials such as pigments and effect substances, do not settle after a prolonged period of time.

A further advantage of the invention is that a preparation, in particular a cosmetic preparation, can be provided, which contains exclusively ingredients which can be derived from plants and/or which are mineral and/or synthetic, but it is completely free from substances which derive from animals.

In order to very substantially minimise mutual interactions between the ingredients used, the basis of this preparation—without the solids phase which is possibly used—should additionally be composed of the smallest possible number of substances.

The preparation according to the invention has advantageous viscoelastic properties which are reflected in the values in respect of complex viscosity and zero viscosity. Thus in accordance with the invention it is possible to obtain compositions whose complex viscosity is in the range of 800 to 6,000 Pa˜s and whose zero viscosity is between 200,000 and 1,200,000 Pa˜s (shearing rate at zero viscosity 0.00005 s⁻¹, temperature 298.15 K; measuring system plate/plate, plates both sand-blasted, plate diameter 25 mm, gap width 1,000 μm; measuring device MCR-301, Anton-Paar; software Rheoplus/32 V6.23).

The above-mentioned preparations according to the invention, because of their excellent durability and coverage, in conjunction with the extremely good application properties, can certainly also be used as camouflage for concealing age spots or rosacea, concealer and the like, also sun protection products involving different sun protection factors (SPF), as far as so-called sun blocks, using very finely divided nanopigments or brightly coloured, more highly pigmented sun blocks which are popular with surfers and windsurfers as body paint. The above-mentioned nanopigments are preferably of a particle size of 5 to 50 nm and can be selected from titanium dioxide, zinc oxide, cerium oxide or aluminium oxide. They are preferably used in amounts of 2 to 20 percent by weight, particularly preferably in amounts of 5 to 10 percent by weight.

The preparation can also be produced without the addition of colouring agents and may optionally contain so-called cosmetic active substances. It is then used as a fixing agent which is applied over a lipstick or a lip rouge. If that uncoloured preparation contains light protection filters, it can be used as lip protection and lip care. As is known, in contrast to the skin of the body, the skin of the lips does not contain any pigmentation. Suitable oil-soluble light filter substances which afford good protection in the UV-A and UV-B range are known in sufficient numbers to the man skilled in the relevant art and are regulated by the respective national or regional legislation for example in the EU, Japan and the U.S.A.—in Germany for example by Appendix 7 to Regulation 3b of the Cosmetics Regulations and they are therefore not to be comprehensively listed here. Therefore, mention will only be made by way of example of isoamyl p-methoxycinnamate as a UV-B filter and 4-methylbenzylidene camphor as a UV-A filter.

The preparation according to the invention is in the form of a soft workable paste which can be easily and uniformly applied and distributed. By virtue of its water content in the inner (disperse phase) upon application to the skin, in conjunction with the volatile silicone oils, it produces a pleasantly cooling effect. It can be removed again from the skin in a manner known to the users—by suitable makeup removal agents or cloths or by washing with fine soap or suitable mild tenside preparations. It can be filled in known manner into suitable vessels such as bottles, possibly with a spatula, pots or tubes, and can be removed again therefrom by the user. However, because of the improved hygiene conditions involved therewith, it can also be introduced into suitable applicator devices, so-called dispenser mechanisms, and applied therefrom. Applicator devices as are known for example from U.S. Pat. No. 6,238,117 or U.S. Pat. No. 6,309,128 present themselves for the application of small amounts as are required for example for application in the region of lips or eyes, as those devices permit very nice fine metering.

The preparation according to the invention can be produced by a procedure whereby the hydrophobic constituents are dissolved or dispersed in the hydrophobic phase, the hydrophilic constituents are dissolved or dispersed in the aqueous phase, emulsifier is added to one of the two phases or both and then the composition is emulsified using shearing force.

Production of the emulsion according to the invention is preferably effected by a procedure whereby firstly the particulate phase is dispersed in the volatile silicone oil, separately therefrom the ester component is melted and the hydrophobic ingredients are added and in a separate operation the aqueous phase is heated to the temperature of the ester component and the hydrophilic ingredients are added. Then all three constituents are brought together, homogenised and then cooled, the emulsion preferably being moved in that case. As soon as the emulsion has approximately reached body temperature, the temperature-sensitive constituents such as fragrances can be added. The finished preparation is finally packaged.

The preparation according to the invention, in particular the cosmetic preparation, will now be described in detail by means of the Examples hereinafter, which however do not definitively describe the invention. In this respect all amounts are stated in percent by weight (% by weight) with respect to the total weight of the preparation, the raw materials are identified by the INCI names which are generally known to the man skilled in the relevant art:

### EXAMPLE 1

### Cream Eyeshadow, Cooling (Non-Transfer)

| | |
|---|---|
| Decamethyl cyclopentasiloxane | 26.300 |
| Pentaerythrityl tetrabehenate | 5.900 |
| Cetyl PEG/PPG-10/1 dimethicone | 3.800 |
| Glycerine | 3.750 |
| Sodium chloride | 0.800 |
| Methylparaben | 0.200 |
| Propylparaben | 0.100 |
| Phenoxyethanol | 0.500 |
| Ascorbyl palmitate | 0.100 |
| Tocopherol | 0.350 |
| Fragrance | 0.300 |
| Titanated mica (C.I. No 77019, 77891) | 9.500 |
| Titanium dioxide (C.I. No 77891) | 3.500 |
| Red iron oxide (C.I. No 77491) | 1.000 |
| Yellow iron oxide (C.I. No 77492) | 0.600 |
| Black iron oxide (C.I. No 77499) | 0.400 |
| Aqua | 42.900 |

Production is effected by a procedure whereby the pigments are put with the silicone oil very intensively in a suitable homogenising machine with an anchor-type agitator and gear ring homogeniser and intensively dispersed by means of the homogeniser. Thereafter the tetrabehenyl tetrabehenate is added together with the emulsifier and melted at about 70-80° C. The parabens, phenoxyethanol, ascorbyl palmitate and tocopherol are now added to the melt. The glycerine and sodium chloride were dissolved in water in a separate vessel and then the water phase was heated to about 70° C. The water phase is now sucked into the homogenisation machine with strong agitation. After the end of the addition the batch is intensively homogenised and then cooled down with strong agitation. At about 45° C. it is briefly homogenised once again, then further cooled with agitation to about 38° C. At that temperature fragrance (the perfume mixture) is added and homogenisation is briefly effected once again. The substance is now deaerated by the application of maximum vacuum and further agitated until a temperature of about 25° C. is reached. It is then discharged and filled into suitable storage containers. That affords a preparation in the form of a light-brown, strongly shiny, water-resistant, workable paste with a zero viscosity of 853,000 Pas.

### EXAMPLE 2

### Cream Eyeshadow, Cooling (Non-Transfer)

| | |
|---|---|
| Hexamethyl siloxane | 25.500 |
| Pentaerythrityl tetrabehenate | 4.600 |
| Sorbitan sesquioleate | 3.800 |
| Butane-1,3-diol | 3.500 |
| Titanated mica (C.I. 77019, 77891) | 15.500 |
| Ultramarine blue (C.I. 77013) | 5.800 |
| Chromium hydroxide green (C.I. 77289) | 1.200 |
| Black iron oxide (C.I. 77499) | 0.800 |
| Tocopherol | 0.500 |
| Fragrance | 0.150 |
| Methylparaben | 0.200 |
| Propylparaben | 0.150 |
| Ascorbyl palmitate | 0.100 |
| Magnesium sulphate | 0.700 |
| Aqua | 37.500 |

Production is effected similarly to the operating procedure described in Example 1. It will be appreciated that care is to be taken to ensure that the pearl pigment, as is familiar to the man skilled in the relevant art, is not exposed to severe shearing forces for too long. The result obtained is a water-resistant, workable paste which is blue with a green cast, with an excellent cooling effect and which can be well applied to the eyelids. The above preparation has a zero viscosity of 451,000 Pas. Experience has shown that somewhat different viscosity values can occur when using other pigment combinations in different amounts.

### EXAMPLE 3

### Lip Rouge (Cooling, Water-Resistant)

| | |
|---|---|
| Hexamethyl disiloxane | 23.500 |
| Pentaerythrityl tetraerucate | 5.200 |
| Trioleyl phosphate | 4.100 |
| Butylene-1,3-glycol | 3.500 |
| Magnesium sulphate | 1.000 |
| Methylparaben | 0.200 |
| Propylparaben | 0.100 |
| Phenoxyethanol | 0.500 |
| Ascorbyl palmitate | 0.100 |
| Tocopherol | 0.300 |
| Fragrance | 0.150 |
| Titanated mica (C.I. No 77019, 77891) | 11.500 |
| Titanium dioxide (C.I. No 77891) | 2.800 |
| Red iron oxide (C.I. No 77491) | 2.200 |
| FD&C Yellow No 5 Al-Lake (C.I. 19140:1) | 0.800 |
| FD&C Red No 3 Al-Lake (C.I. 45430:1) | 0.700 |
| Aqua | 43.350 |

Production is effected similarly to the above-described mode of operation. The result obtained is a powerfully red preparation with a strong sheen, in the form of a water-resistant, workable paste with a zero viscosity of 491,000 Pas, which is particularly well suited for the automatic applicator devices described hereinbefore.

### EXAMPLE 4

### Lip Rouge with Sun Protection Filter (Cooling, Water-Resistant)

| | |
|---|---|
| Decamethyl cyclopentasiloxane | 22.500 |
| Dodecamethyl cyclohexasiloxane | 3.500 |
| Pentaerythrityl tetraerucate | 4.800 |
| Polyglyceryl-2-PEG-4 isostearate | 3.800 |
| Glycerine | 3.700 |
| Sodium chloride | 0.600 |
| Methylparaben | 0.200 |
| Propylparaben | 0.150 |
| Phenoxyethanol | 0.600 |
| Ascorbyl palmitate | 0.100 |
| Tocopherol | 0.250 |
| Fragrance | 0.150 |
| Titanated mica (C.I. 77019, 77891) | 7.000 |
| Titanium dioxide nanopigment (C.I. 77891) | 6.000 |
| Isoamyl p-methoxycinnamate | 1.500 |
| 4-Methylbenzylidene camphor | 2.000 |
| Red iron oxide (C.I. 77491) | 2.000 |
| Titanium dioxide (C.I. 77891) | 2.500 |
| D&C Red No 6, Ba-Lake (C.I. 15850:2) | 3.500 |
| Aqua | 35.150 |

Production is effected similarly to the above-described Examples. The result obtained is a workable paste which is coloured with a powerful red and with a fine pearl sheen, for protecting the lips from strong sunshine. It has a zero viscosity in the region of 386,000 Pas.

### EXAMPLE 5

### Sunblock for Surfers (Water Resistant)

| | |
|---|---|
| Hexamethyl disiloxane | 24.000 |
| Phenyltrimethicone | 0.800 |
| Pentaerythrityl tetrabehenate | 4.800 |
| Pentaerythrityl tetra-(12-hydroxy)-stearate | 1.000 |
| Sorbitan sesquioleate | 4.200 |
| Iron oxide red (C.I. No 77491) | 3.200 |
| Iron oxide yellow (C.I. No 77492) | 1.800 |
| Propane-1,2-diol | 3.800 |
| Titanium dioxide (nanopigment) | 10.000 |
| Iron oxides (red and yellow) | 5.000 |
| Polyester-3, orange 5 | 7.500 |
| Isoamyl p-methoxycinnamate | 3.500 |
| 4-Methylbenzylidene camphor | 2.500 |
| Tocopherol | 0.600 |
| Fragrance | 0.200 |
| Methylparaben | 0.200 |
| Propylparaben | 0.150 |
| Ascorbyl palmitate | 0.100 |
| Sodium chloride | 0.600 |
| Aqua | 26.050 |

Production is effected similarly to the above-described mode of operation. The result obtained is a preparation in the form of a water-resistant, workable paste with an intensively orange-yellow colouring and a zero viscosity of 780,000 Pas.

### EXAMPLE 6

### Agent for Fixing Lipstick and Lip Rouge (Wipe-Resistant)

| | |
|---|---|
| Decamethyl cyclopentasiloxane | 32.500 |
| Phenyltrimethicone | 0.500 |
| Pentaerythrityl tribehenate | 6.200 |
| Sorbitan sesquioleate | 4.750 |
| Tocopherol | 0.300 |
| Diglycerine | 3.000 |
| Magnesium stearate | 2.800 |
| Fragrance | 0.200 |
| Methylparaben | 0.200 |
| Propylbaraben | 0.150 |
| Ascorbyl palmitate | 0.100 |
| Magnesium sulphate | 0.600 |
| Aqua | 48.700 |

Production is effected similarly to the foregoing Examples—here the magnesium stearate is dispersed intensively in silicone oil—otherwise the procedure is as described in Example 1. The result obtained is a white creamy preparation which leaves behind on the lips a transparent film with a pleasant cooling effect. The preparation has a zero viscosity of 685,0

1-43. (canceled)

44. A preparation in the form of an emulsion comprising a lipophilic outer phase and a hydrophilic inner phase, wherein the outer phase comprises at least one polyvalent ester, at least one volatile silicone and optionally further lipophilic additives, and the inner phase contains an aqueous medium and optionally hydrophilic additives, wherein the preparation further includes at least one emulsifier and a solids phase, wherein the polyvalent ester is selected from the group consisting of (1) polyvalent, at least divalent alcohols each with at least two acid residues, (2) polyvalent, at least divalent acids and at least two respective alcohol residues, and (3) polyvalent alcohols and polyvalent acids, wherein the chain length of the residues originating from the alcohol is C₂ to C₆₀ and the chain length of the residues originating from acids is C₄ to C₆₀, wherein the ester has a melting point in the range of 40 to 200° C.

45. A preparation, in particular a cosmetic preparation according to claim 44, wherein the polyvalent ester has further functional groups selected from the group consisting of hydroxyl, carboxyl, amino, acid amide, ester groups and mixtures thereof.

46. A preparation according to claim 44, wherein the polyvalent ester component contains a compound formula I: 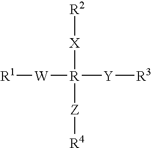
wherein R is a linear, branched or cyclic hydrocarbon residue with 1 to 8 carbon atoms, W, X, Y, Z are each independently of each other —C(O)O—,
—OC(O)—, —O—, —NR⁵₂ or —NC(O)— and each of the residues R¹, R², R³, R⁴ and R⁵ respectively independently denotes a linear or branched, long-chain hydrocarbon residue.

47. A preparation according to claim 46, wherein the polyvalent ester is a compound of formula I, wherein W, X, Y and Z each signify an ester group, R signifies C and at least three of the residues R¹, R², R³ and R⁴ respectively independently signify a C₆ to C₂₄ alkyl residue and the fourth of the residues signifies H or a C₆ to C₂₄ alkyl residue.

48. A preparation according to claim 44, wherein in the ester component the sum of the carbon atoms of alcohol and carboxylic acid residues is in a range of 35 to 150.

49. A preparation according to claim 44, wherein the polyvalent ester is a pentaerythritol ester.

50. A preparation according to claim 49, wherein the pentaerythritol ester is selected from the group consisting of pentaerythrityl tetramyristate, tristearate, tetrastearate, triisostearate, tetraisostearate, tribehenate, tetrabehenate, tetra-(ethylhexyl-dodecanoate), tri-(12-hydroxy)-stearate, tetra-(12-hydroxy)-stearate, trierucate, tetraerucate, tetramelissinate and mixtures thereof.

51. A preparation according to claim 48, wherein the ester is selected from the group consisting of behenyl behenate, behenyl mellisinate, isostearylethylhexyl dodecanoate and mixtures thereof.

52. A preparation according to claim 44, wherein the preparation includes exclusively ingredients which can be derived from plants and/or which are mineral and/or synthetic and in that respect is completely free from substances which derive from animals.

53. A preparation according to claim 44, further including a wax with a dropping point of between 50 and 200° C.

54. A preparation according to claim 44, further including a wax with a dropping point of between 60 and 150° C.

55. A preparation according to claim 44, further including a wax with a dropping point of between 75 and 120° C.

56. A preparation according to claim 50, wherein the wax has both at least one alcohol residue and also at least one carboxylic acid residue which has a saturated or singly or multiply unsaturated straight-chain or branched hydrocarbon component.

57. A preparation according to claim 56, wherein the wax is a mixture of candellila wax and carnauba wax.

58. A preparation according to claim 44, wherein the polyvalent ester is contained in a range of between 0.5 and 20 percent by weight.

59. A preparation according to claim 44, wherein the polyvalent ester is contained in a range of between 2 and 12 percent by weight.

60. A preparation according to claim 44, wherein the volatile silicone oil is selected from the group consisting of hexamethyl cyclotrisiloxane, octamethyl cyclotetrasiloxane, decamethyl cyclopentasiloxane, dodecamethyl cyclohexasiloxane, hexamethyl disiloxane, octamethyl trisiloxane, decamethyl tetrasiloxane, dodecamethyl pentasiloxane and mixtures thereof.

61. A preparation according to claim 44, further including a non-volatile silicone oil in an amount of less than 5 percent by weight.

62. A preparation according to claim 44, wherein the silicone oil is a non-volatile silicone oil or mixtures of non-volatile silicone oils.

63. A preparation according to claim 61, wherein the non-volatile silicone oil is selected from the group consisting of dimethyl polysiloxanes with differing chain length and differing viscosity, and arylated silicone oils selected from the group consisting of phenyldimethicone, phenyltrimethicone, diphenyldimethicone and mixtures thereof.

64. A preparation according to claim 44, wherein the emulsifier is a W/O emulsifier or a mixture of W/O emulsifier and W/S emulsifier.

65. A preparation according to claim 44, wherein the emulsifier is a non-ionogenic W/O emulsifier.

66. A preparation according to claim 65, wherein the non-ionogenic W/O emulsifier is selected from the group consisting of sorbitan sesquioleate, sorbitan laurate, soya sterol, PEG-5 soya sterol, polyglyceryl-4 isostearate, polyglyceryl-2-PEG-4 isostearate, polyglyceryl-2 sesquiisostearate, cetyl-PEG/PPG dimethicone, trioleyl phosphate, trioleth-8-phosphate, trilaureth-4-phosphate and mixtures thereof.

67. A preparation according to claim 44, wherein a stabilisation agent is included.

68. A preparation according to claim 67, wherein the stabilisation agent is an inorganic salt which is soluble in a water phase.

69. A preparation according to claim 68, wherein the inorganic salt is selected from the group consisting of sodium chloride, potassium chloride, sodium sulphate, magnesium sulphate and mixtures thereof.

70. A preparation according to claim 67, wherein the stabilisation agent is contained in an amount of 0.05 to 3 percent by weight in the water phase.

71. A preparation according to claim 67, wherein the stabilisation agent is contained in an amount of 0.3 to 2 percent by weight in the water phase.

72. A preparation according to claim 67, wherein an agent for keeping the preparation moist is additionally contained in the water phase.

73. A preparation according to claim 72, wherein the moistening agent is selected from the group consisting of propylene glycol, dipropylene glycol, tripropylene glycol, butylene glycol, glycerine, diglycerine, triglycerine, sorbitol, mannitol, xylitol, glucose, fructose, sucrose, carbamide (urea), lactic acid, citric acid, pyrrolidone carboxylic acid (PCA) or salts of said acids, and mixtures thereof.

74. A preparation according to one of claims 72, wherein the agent is contained in the water phase in an amount of 0.1 to 5 percent by weight.

75. A preparation according to one of claims 72, wherein the agent is contained in the water phase in an amount of 1 to 3 percent by weight.

76. A preparation according to claim 44, wherein the solid phase comprises fillers, effect substances, inorganic pigments, organic pigments and mixtures thereof.

77. A preparation according to claim 76, wherein the filler is selected from the group consisting of talcum, kaolin, starch, modified starch, polytetrafluoroethylene powder, nylon powder, boronitride, Mg stearate, Ca stearate, Sr stearate, Zn stearate, and mixtures thereof.

78. A preparation according to claim 76, wherein the solid phase is contained in quantitative proportions in a range of 0 to 40 percent by weight.

79. A preparation according to claim 76, wherein the solid phase is contained in quantitative proportions in a range of 5 to 30 percent by weight.

80. A preparation according to claim 76, wherein the solid phase is contained in quantitative proportions in a range of 8 to 20 percent by weight.

81. A preparation according to claim 76, wherein the inorganic pigment is a nanopigment with a particle size of 5 to 50 nm, which is selected from the group consisting of titanium dioxide, zinc oxide, zirconium oxide, cerium oxide, aluminium oxide, silicon dioxide, and mixtures thereof.

82. A preparation according to claim 81, wherein the nanopigment is contained in an amount of 2 to 20 percent by weight.

83. A preparation according to claim 81, wherein the nanopigment is contained in an amount of 5 to 10 percent by weight.

84. A preparation according to claim 81, wherein the nanopigment is combined with oil-soluble UV-A and UV-B light filter substances.

85. A preparation according to claim 84, wherein the oil-soluble UV-A and UV-B light filter substances are 4-methylbenzylidene camphor and isoamyl p-methoxycinnamate.

86. A preparation according to claim 76, wherein the inorganic pigment is selected from the group consisting of titanium dioxide, zinc oxide, iron oxides, chrome oxide, hydrated chrome oxide, ultramarine, Berlin Blue (Ferric Blue), mica, mica coated with titanium dioxide, mica coated with titanium dioxide and metal oxides, bismuth oxide chloride, coated bismuth oxide chloride, metal powder in flake form of aluminium, brass, bronze, copper, silver, gold, and mixtures thereof.

87. A preparation according to claim 76, wherein the organic pigment comprise organic colouring agents with at least one of aluminium, barium, calcium, strontium and zirconium.

88. A preparation according to claim 44, wherein the preparation it is suitable in the area of decorative cosmetics for caring for, colouring and improving skin, lips and eyelids.

89. A preparation according to claim 88, wherein the cosmetic preparation is a lip rouge, blusher, makeup, eyeshadow, camouflage or a concealer.

90. A preparation according to claim 88, wherein the cosmetic preparation is an agent for fixing lipstick or lip rouge, a care foundation, a skin care agent or a sun protection agent.

91. A preparation according to claim 44, wherein the preparation is a workable paste in the form of a water-in-silicone emulsion with a complex viscosity of 800 to 6,000 Pas and a zero viscosity of 200,000 to 1,200,000 Pas, (shearing rate at zero viscosity 0.00005 s⁻¹; temperature 298.15 K).

92. A process for the production of a preparation according to claim 44, wherein the particulate phase is dispersed in the volatile silicone, separately therefrom the ester component is melted and the hydrophobic ingredients are added, in a separate operation the aqueous phase is heated to the temperature of the ester component and the hydrophilic ingredients are added, then all three constituents are brought together, homogenised and then cooled and possibly as soon as the emulsion has approximately reached body temperature temperature-sensitive constituents such as fragrances are added.
